(19) United States
(12) Patent Application Publication
(10) Pub. No.: US 2026/0337660 A1
(43) Pub. Date: Oct. 29, 2026

(54) METHOD OF PREDICTION OF GESTATIONAL DIABETES MELLITUS BASED ON THE EXPRESSION PROFILE OF CARDIOVASCULAR MIRNAS

(71) Applicant: GENESPECTOR S.R.O., Praha (CZ)

(72) Inventor: Ilona HROMADNIKOVA, Praha (CZ)

(21) Appl. No.: 19/102,490

(22) PCT Filed: Mar. 8, 2023
(86) PCT No.: PCT/CZ2023/050047

§ 371 Date: Feb. 10, 2025

(30) Foreign Application Priority Data
Aug. 12, 2022 (CZ) PV 2022-335

(51) Int. Cl. C12Q 1/6809

(52) U.S. Cl. CPC C12Q 1/6809; C12Q 2600/112; C12Q 2600/118; C12Q 2600/158; C12Q 2600/178; G01N 2800/042; G01N 2800/50; G01N 2800/60

(57) ABSTRACT

Method of prediction of gestational diabetes mellitus including both its milder and more severe form. Pregnant women are screened to determine the expression profile of two or more miRNAs in whole peripheral venous blood collected in the period of 10th-13th gestational week, whereas said two or more miRNAs are selected from the group miR-1-3p, miR-20a-5p, miR-20b-5p, miR-23a-3p, miR-100-5p, miR-125b-5p, miR-126-3p, miR-181a-5p, miR-195-5p, miR-499a-5p, and miR-574-3p.

### TECHNICAL FIELD

The invention relates to the field of analysis of non-coding nucleic acids and expression markers, specifically microRNAs (miRNAs), using molecular-biological methods, primarily quantitative polymerase chain reaction with reverse transcription (RT-qPCR), and their use in screening to predict the development of metabolic disorders in pregnancy.

### BACKGROUND ART

Gestational diabetes mellitus (GDM), also known as gestational diabetes, is a metabolic disorder of the mother manifested by glucose intolerance, which appears during pregnancy and spontaneously resolves during the postpartum period. Without appropriate diabetological prenatal care in the form of diet modification, or targeted therapy in more serious cases, GDM increases the risk of pregnancy complications in the mother, newborn morbidity, and also has long-term consequences for both the mother and the child in the form of the risk of developing other metabolic disorders, for example obesity or type 2 diabetes mellitus. In clinical practice, GDM is diagnosed through an oral glucose tolerance test, which is performed at the turn of the second and third trimester. However, this test only detects already developed GDM. Therefore, the effort of contemporary medicine is to predict this metabolic disorder in time and, ideally, to prevent its occurrence or at least to mitigate its course and effects by an early intervention.

Methods for predicting the onset of GDM in the mother during the first trimester are known from prior art, for example from documents Syngelaki et al., Fetal. Diagn. Ther. 2015, 38, p 14-21; Sweeting et al., Diabetes Res. Clin. Pract. 2017, 127, p 44-50; Sweeting et al., Fetal Diagn. Ther. 2019, 45, p 76-84.; Wang et al., Diabetes Res. Clin. Pract. 2018, 142, p 130-138.; Sakurai et al., J. Diabetes Investig. 2019, 10, p 513-520; Tenenbaum-Gavish et al., Placenta 2020, 101, p 80-89; Zheng et al., Front. Public Health 2022, 10, p 850191; Zhang et al., Exp. Ther. Med. 2020, 20, p 293-300; Cremona et al., Clin. Nutr. ESPEN2021, 45, p 312-321; Shaarbaf Eidgahi et al., BMC Pregnancy Childbirth 2022, 22, p 13. However, these methods are primarily based on data about the mother and her clinical parameters, such as age, weight, height, race, amount of subcutaneous fat, blood pressure, family history of diabetes, smoking, physical activity, use of medications to promote ovulation, or the occurrence of GDM in previous pregnancies. In the case of some of the above-mentioned methods, these data are only supplemented by a blood count and the determination of an incomplete set of biochemical markers, for example coagulation factors, indicators of glycolipid metabolism, serum glutamine, ethanolamine and 1,3-diphosphoglycerate in urine, the soluble form of the CD163 receptor, tumor necrosis factor α (TNF-α), placental protein 13 (PP13), or pregnancy-associated plasma protein A (PAPP-A). The prediction of the risk of the onset of GDM by these methods relies on the above-mentioned data about the mother, which can often be incomplete, influenced by the mother's current physical or psychological state, or inaccurately or even falsely stated. Thus, this degree of subjectivity represents a fundamental deficiency of the state of the art significantly affecting the accuracy of prediction in real clinical conditions. At the same time, using methods known from prior art, it is not possible to make a prediction at all without knowledge of this information, so it is not possible to reliably analyze anonymous samples or to evaluate samples in a laboratory on a mass scale. Another shortcoming of this state of the art is the use of non-specific biochemical markers, the values of which can vary significantly within the population, which further complicates the degree of reliability of the prediction.

Examples of changes in the levels of selected circulating microRNAs (miRNAs) associated with the onset of GDM have been described in scientific literature. Lamadrid-Romero et al., Neurosci. Res. 2018, 130, p 8-22 describes changes in levels of miRNAs associated with function of nervous system in women with GDM in the second and third trimesters compared with first-trimester values. However, these findings cannot be used to predict the onset of GDM. Documents Thamotharan et al., PLoS ONE 2022, 17(5), e0267564; Yoffe et al., Eur. J. Endocrinol. 2019, 181, p 565-577; Légaré et al., BMJ Open Diabetes Res. Care 2022, 10, e002703; Cao et al., J. Obstet. Gynaecol. Res. 2017, 43, p 974-981; Sorensen et al., Cells 2021, 10, p 170; Zhao et al., PLoS One 2011, 6, e23925 describe several dozen miRNAs whose expression profile is altered during the first trimester in women who subsequently develop GDM, and mention the possibility of using these findings to predict the onset of GDM. However, the documents do not discuss the statistical significance of individual miRNAs, which is necessary to conclude which miRNAs are suitable for such a prediction in clinical practice. In addition, the cited documents describe markers circulating freely in serum or plasma or as a part of plasma exosomes. The processing of plasma exosomes is a demanding process that is not suitable for wider use in clinical practice.

Document Hromadnikova et al., Int. J. Mol. Sci. 2020, 21, p 2437 describes the postpartum, i.e., after-childbirth, profile of selected miRNAs in women who experienced GDM during pregnancy. However, since the observed changes in miRNA expression profiles were caused by a metabolic disorder that only develops in the later stages of pregnancy, it is not possible to use the knowledge known from the cited document for the development of a method that would predict the onset of GDM during the first trimester of pregnancy, i.e., in the period of 10th to 13th gestational week. In other words, the increased or decreased values of the levels of given miRNAs after experiencing a pregnancy metabolic disorder do not correspond in any way to their values before the onset of the disorder and are completely independent of them. Since the findings from the cited document are based on changes caused by experiencing this metabolic disorder, using these changes to predict the disorder before it occurs is not possible.

Documents Hromadnikova et al., Biomedicines 2022, 10, p 256; Hromadnikova et al., Biomedicines 2022, 10, p 718; Hromadnikova et al., Int. J. Mol. Sci. 2022, 23, p 3951 describe methods for predicting pregnancy complications in the form of preeclampsia, fetal growth restriction, gestational hypertension, small fetal size for a given gestational age, and preterm birth (in the form of spontaneous preterm birth or preterm premature rupture of membranes), as well as detection of undiagnosed chronic hypertension, using selected cardiovascular miRNAs as biomarkers. However, since the aforementioned complications are associated with impaired placental function and abnormal activity of the mother's cardiovascular system, it is not possible to use the knowledge known from the cited documents, i.e., the use of cardiovascular miRNAs, in an obvious way for the development of a method for predicting the onset of GDM, which is a metabolic disorder, and not a complication associated with function of the placenta or the cardiovascular system of the mother.

Goal of the present invention is to eliminate the drawbacks of the prior art by developing a method that can be easily implemented on a large scale even in a commonly equipped molecular-genetics laboratory and which is able to predict—already during the first trimester of pregnancy—in a robust manner and with high reliability the onset of gestational diabetes mellitus in the later stages of pregnancy, for which there is no possibility of prediction in current clinical practice, and to do so even with anonymous samples, i.e., without the need to know the data about the mother and her clinical parameters.

### SUMMARY OF INVENTION

The present invention is based on determining levels of 11 specific miRNAs (miR-1-3p, miR-20a-5p, miR-20b-5p, miR-23a-3p, miR-100-5p, miR-125b-5p, miR-126-3p, miR-181a-5p, miR-195-5p, miR-499a-5p, miR-574-3p) by an RT-qPCR method in samples of whole peripheral venous blood taken from pregnant women during the standard first-trimester screening, i.e., in the period of 10th to 13th gestational week. This way, it is possible to predict the onset of GDM with high probability and reliability. By selecting suitable subsets from this group of miRNAs, it is further possible to specify whether it is going to be a milder form requiring only dietary modifications, or a more severe form requiring therapeutic intervention in addition to dietary modifications, for example in the form of medication.

Although the connection between changes in levels of the above-mentioned miRNAs and experienced GDM or the prediction of pregnancy complications associated with abnormal function of the placenta and the mother's cardiovascular system has already been presented in scientific documents, and is therefore known from the prior art, their use for predicting GDM is not obvious or simply deducible from these documents due to the large number of described miRNAs, their specific connection with the cardiovascular system and not metabolism, the high variability of possible changes in expression levels, and the necessity of non-obvious selection of a specific subset of 11 of these markers.

The method was developed based on analyses of selected samples from 4,187 women in the first trimester of pregnancy, statistical processing of the results of these analyses, and their comparison with the subsequent course of pregnancy in the monitored women. A complete health status during the entire pregnancy, including all complications and the course of childbirth, was known in 3,028 of these women. Of these 3,028, 121 women developed GDM without any other pregnancy complication. Of these 121, 101 women had a milder form of GDM requiring only dietary modifications and 20 women had a more severe form requiring therapeutic intervention in addition to dietary modifications.

The method according to the invention is carried out as follows. First, the collected blood is processed into a leukocyte lysate. The contained RNA is then extracted and short RNAs are subsequently isolated. Resulting purified solution is analyzed using a two-step RT-qPCR reaction in a device maintaining ideal temperature conditions for individual steps that are repeated cyclically in the presence of standard and sequence-specific chemicals, namely miRNA-specific stem-loop RT primers, miRNA-specific forward and reverse PCR primers, and miRNA-specific MGB probes. In each step, a fluorescence signal released from the probe is measured and the cycle is usually repeated 40-45 times in total. After finishing the program, Ct values are read in the individual channels and the results correspond to the original number of monitored nucleic acid molecules. RNA extraction from leukocyte lysate and RT-qPCR analysis are standards in current molecular diagnostic practice. The procedures are fast, simple to perform, and allow for easy automation. Therefore, this diagnostic method is suitable for implementation in most genetic laboratories and for wide use in clinical practice.

The normal distribution of expression of selected miRNAs was determined based on a sample of women who had a physiological pregnancy without complications. These levels were subsequently compared with miRNA levels in samples obtained from women who developed GDM during pregnancy, and the results were statistically processed using the Kruskal-Wallis test and the Mann-Whitney test. Based on the results, a specific set of 11 miRNAs (miR-1-3p, miR-20a-5p, miR-20b-5p, miR-23a-3p, miR-100-5p, miR-125b-5p, miR-126-3p, miR-181 a-5p, miR-195-5p, miR-499a-5p, miR-574-3p) was selected. These miRNAs are all up-regulated before the onset of GDM and determination of their levels enables a prediction of GDM with a sufficiently high sensitivity at a sufficiently low rate of false positives. A generally accepted value is 10% false positive rate. Out of this set of 11 miRNAs, determination of 8 specific miRNAs (miR-1-3p, miR-20a-5p, miR-20b-5p, miR-100-5p, miR-125b-5p, miR-195-5p, miR-499a-5p, miR-574-3p) allows to specify whether it is going to be a milder form requiring only dietary modifications and determination of 3 specific miRNAs (miR-20a-5p, miR-20b-5p, miR-195-5p) allows to specify whether it is going to be a more severe form requiring therapeutic intervention in addition to dietary modifications.

As can be seen from results presented below, the method makes it possible to effectively predict GDM based solely on the results of analysis of a miRNA profile in peripheral venous blood, i.e., without the need for additional clinical examination of women or knowledge of their medical history. Thus, the method is highly objective without the possibility of its distortion by false or erroneous data, and at the same time enables the testing of samples on a large scale, including their evaluation, while preserving the anonymity of the patients. Nevertheless, the method also allows a combination with the clinical characteristics of the mother, which increases the probability of a successful prediction even further.

### Gestational Diabetes Mellitus (GDM)

In women who developed GDM during pregnancy, monitoring the up-regulation of the following 11 miRNAs in whole peripheral venous blood collected during the first trimester proved to be the most suitable for prediction: miR-1-3p, miR-20a-5p, miR-20b-5p, miR-23a-3p, miR-100-5p, miR-125b-5p, miR-126-3p, miR-181 a-5p, miR-195-5p, miR-499a-5p, miR-574-3p. Using this method, without any additional clinical examination of the women or knowledge of their medical history, it was possible to predict 47.9% of cases at 10% false positive rate (FPR). By combining these 11 miRNAs with three basic clinical characteristics of the mother (age in early pregnancy, body mass index (BMI) in early pregnancy, infertility treatment with assisted reproduction methods), it was possible to predict 69.2% of cases at 10% FPR. By combining these 11 miRNAs with seven clinical characteristics of the mother (age in early pregnancy, BMI in early pregnancy, infertility treatment with assisted reproduction methods, maternal history of miscarriage, presence of thrombophilic gene mutations, positive result of first-trimester prenatal screening for early preeclampsia and spontaneous preterm birth before the 34th week of gestation and fetal growth restriction before the 37th week of gestation using an established method of a predictive routine algorithm within a computer application for obstetrics and gynecology databases, the incidence of diabetes mellitus in close first-degree relatives) it was possible to predict 72.5% cases at 10% FPR.

### Milder Form of GDM Requiring Only Dietary Modifications

In women who developed a milder form of GDM during pregnancy, monitoring the up-regulation of the following 8 miRNAs in whole peripheral venous blood collected during the first trimester proved to be the most suitable for prediction: miR-1-3p, miR-20a-5p, miR-20b-5p, miR-100-5p, miR-125b-5p, miR-195-5p, miR-499a-5p, miR-574-3p. Using this method, without any additional clinical examination of the women or knowledge of their medical history, it was possible to predict 34.7% of cases at 10% FPR. By combining these 8 miRNAs with three basic clinical characteristics of the mother (age in early pregnancy, BMI in early pregnancy, infertility treatment with assisted reproduction methods), it was possible to predict 50.5% of cases at 10% FPR. By combining these 8 miRNAs with seven clinical characteristics of the mother (age in early pregnancy, BMI in early pregnancy, infertility treatment with assisted reproduction methods, maternal history of miscarriage, presence of thrombophilic gene mutations, positive result of first-trimester prenatal screening for early preeclampsia and spontaneous preterm birth before the 34th week of gestation and fetal growth restriction before the 37th week of gestation using an established method of a predictive routine algorithm within a computer application for obstetrics and gynecology databases, the incidence of diabetes mellitus in close first-degree relatives) it was possible to predict 56.4% cases at 10% FPR.

### More Severe Form of GDM Requiring Therapeutic Intervention in Addition to Dietary Modifications

In women who developed a more severe form of GDM during pregnancy, monitoring the up-regulation of the following 3 miRNAs in whole peripheral venous blood collected during the first trimester proved to be the most suitable for prediction: miR-20a-5p, miR-20b-5p, miR-195-5p. In these cases, no significant up-regulation of miR-1-3p, miR-100-5p, miR-125b-5p, miR-499a-5p, miR-574-3p was observed, as these are significantly up-regulated only in the milder form of GDM. Using this method, without any additional clinical examination of the women or knowledge of their medical history, it was possible to predict 30.0% of cases at 10% FPR. By combining these 3 miRNAs with three basic clinical characteristics of the mother (age in early pregnancy, BMI in early pregnancy, infertility treatment with assisted reproduction methods), it was possible to predict 79.0% of cases at 10% FPR. By combining these 3 miRNAs with seven clinical characteristics of the mother (age in early pregnancy, BMI in early pregnancy, infertility treatment with assisted reproduction methods, maternal history of miscarriage, presence of thrombophilic gene mutations, positive result of first-trimester prenatal screening for early preeclampsia and spontaneous preterm birth before the 34th week of gestation and fetal growth restriction before the 37th week of gestation using an established method of a predictive routine algorithm within a computer application for obstetrics and gynecology databases, the incidence of diabetes mellitus in close first-degree relatives) it was possible to predict 89.5% cases at 10% FPR.

### BRIEF DESCRIPTION OF DRAWINGS

FIG. 1 depicts ROC (Receiver Operating Characteristic) curve obtained from the statistical analysis of the up-regulation of 11 selected miRNAs (miR-1-3p, miR-20a-5p, miR-20b-5p, miR-23a-3p, miR-100-5p, miR-125b-5p, miR-126-3p, miR-181a-5p, miR-195-5p, miR-499a-5p, miR-574-3p) for prediction of GDM.

FIG. 2 depicts ROC curve obtained from the statistical analysis of the up-regulation of 11 selected miRNAs (miR-1-3p, miR-20a-5p, miR-20b-5p, miR-23a-3p, miR-100-5p, miR-125b-5p, miR-126-3p, miR-181 a-5p, miR-195-5p, miR-499a-5p, miR-574-3p) in a combination with three basic clinical characteristics of the mother (age in early pregnancy, BMI in early pregnancy, infertility treatment with assisted reproduction methods) for prediction of GDM.

FIG. 3 depicts ROC curve obtained from the statistical analysis of the up-regulation of 11 selected miRNAs (miR-1-3p, miR-20a-5p, miR-20b-5p, miR-23a-3p, miR-100-5p, miR-125b-5p, miR-126-3p, miR-181 a-5p, miR-195-5p, miR-499a-5p, miR-574-3p) in a combination with seven clinical characteristics of the mother (age in early pregnancy, BMI in early pregnancy, infertility treatment with assisted reproduction methods, maternal history of miscarriage, presence of thrombophilic gene mutations, positive result of first-trimester prenatal screening for early preeclampsia and spontaneous preterm birth before the 34th week of gestation and fetal growth restriction before the 37th week of gestation using an established method of a predictive routine algorithm within a computer application for obstetrics and gynecology databases, the incidence of diabetes mellitus in close first-degree relatives) for prediction of GDM.

FIG. 4 depicts ROC curve obtained from the statistical analysis of the up-regulation of 8 selected miRNAs (miR-1-3p, miR-20a-5p, miR-20b-5p, miR-100-5p, miR-125b-5p, miR-195-5p, miR-499a-5p, miR-574-3p) for prediction of a milder form of GDM requiring only dietary modifications.

FIG. 5 depicts ROC curve obtained from the statistical analysis of the up-regulation of 8 selected miRNAs (miR-1-3p, miR-20a-5p, miR-20b-5p, miR-100-5p, miR-125b-5p, miR-195-5p, miR-499a-5p, miR-574-3p) in a combination with three basic clinical characteristics of the mother (age in early pregnancy, BMI in early pregnancy, infertility treatment with assisted reproduction methods) for prediction of a milder form of GDM requiring only dietary modifications.

FIG. 6 depicts ROC curve obtained from the statistical analysis of the up-regulation of 8 selected miRNAs (miR-1-3p, miR-20a-5p, miR-20b-5p, miR-100-5p, miR-125b-5p, miR-195-5p, miR-499a-5p, miR-574-3p) in a combination with seven clinical characteristics of the mother (age in early pregnancy, BMI in early pregnancy, infertility treatment with assisted reproduction methods, maternal history of miscarriage, presence of thrombophilic gene mutations, positive result of first-trimester prenatal screening for early preeclampsia and spontaneous preterm birth before the 34th week of gestation and fetal growth restriction before the 37th week of gestation using an established method of a predictive routine algorithm within a computer application for obstetrics and gynecology databases, the incidence of diabetes mellitus in close first-degree relatives) for prediction of a milder form of GDM requiring only dietary modifications.

FIG. 7 depicts ROC curve obtained from the statistical analysis of the up-regulation of 3 selected miRNAs (miR-20a-5p, miR-20b-5p, miR-195-5p) for prediction of a more severe form of GDM requiring therapeutic intervention in addition to dietary modifications.

FIG. 8 depicts ROC curve obtained from the statistical analysis of the up-regulation of 3 selected miRNAs (miR-20a-5p, miR-20b-5p, miR-195-5p) in a combination with three basic clinical characteristics of the mother (age in early pregnancy, BMI in early pregnancy, infertility treatment with assisted reproduction methods) for prediction of a more severe form of GDM requiring therapeutic intervention in addition to dietary modifications.

FIG. 9 depicts ROC curve obtained from the statistical analysis of the up-regulation of 3 selected miRNAs (miR-20a-5p, miR-20b-5p, miR-195-5p) in a combination with seven clinical characteristics of the mother (age in early pregnancy, BMI in early pregnancy, infertility treatment with assisted reproduction methods, maternal history of miscarriage, presence of thrombophilic gene mutations, positive result of first-trimester prenatal screening for early preeclampsia and spontaneous preterm birth before the 34th week of gestation and fetal growth restriction before the 37th week of gestation using an established method of a predictive routine algorithm within a computer application for obstetrics and gynecology databases, the incidence of diabetes mellitus in close first-degree relatives) for prediction of a more severe form of GDM requiring therapeutic intervention in addition to dietary modifications.

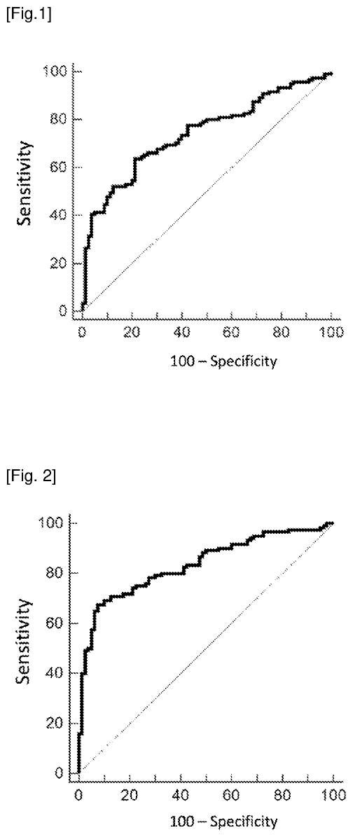

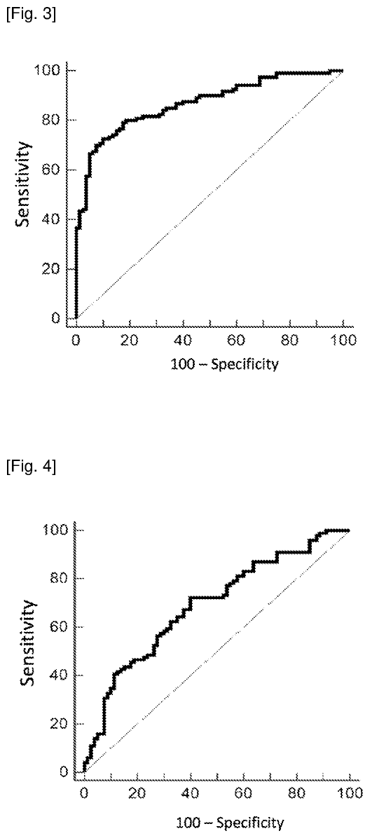

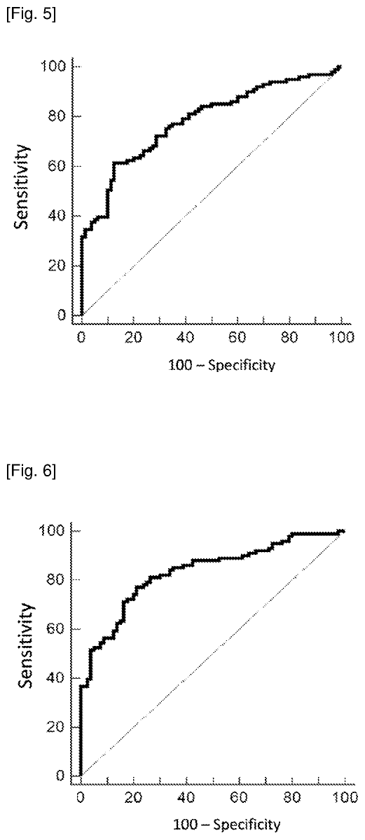

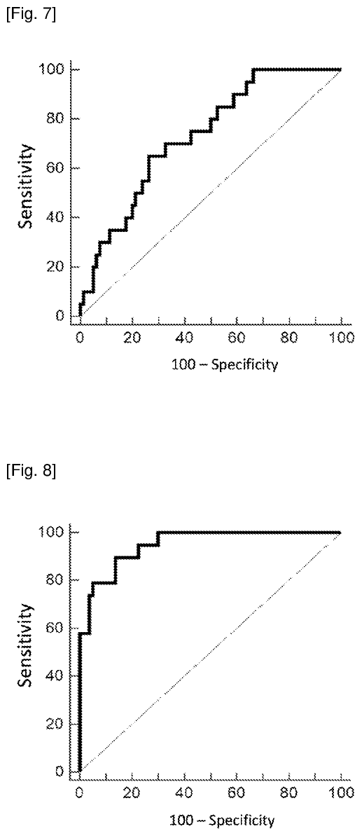

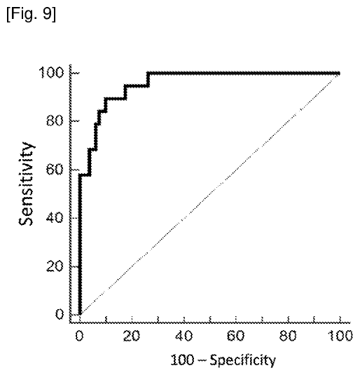

### DESCRIPTION OF EMBODIMENTS

### Example 1

Example 1 describes a general implementation of RT-qPCR analysis determining the amount of miRNA in a tested sample of whole peripheral venous blood and in a reference sample normalized to a simultaneously determined amount of selected endogenous controls (RNU58A a RNU38B).

RNA isolation is performed from thawed leukocyte lysate using a mixture of acidic phenol and chloroform. Long RNAs are further removed from the obtained RNA and short RNAs are concentrated using a column with a glass fiber filter and ethanol of different concentrations in the individual isolation steps. The isolated RNA containing short RNAs is directly used as a template in a two-step RT-qPCR reaction. Reverse transcription takes place under the following conditions: 30 min at 16 °C, 30 min at 42 °C, and 5 min at 85 °C. This is followed by a polymerase chain reaction under the following conditions: 50 °C for 2 min, 95 °C for 10 min, then cycling at 95 °C for 15 s and 60 °C for 1 min. After each cycle, the fluorescence is measured in the FAM and ROX channels (passive reference for fluorescence normalization). The cycling is repeated a total of 40 to 45 times. After the program ends, Ct values in the individual channels are read. Within one sample, the Ct value in the FAM channel is obtained for the measured miRNA or for the short RNA serving as an endogenous control. These values correspond to the expression of individual genes in the biological sample. The normalized expression value is obtained by subtracting the Ct value of the endogenous control (geometric mean of RNU58A and RNU38B) from the Ct value of the miRNA in the assayed sample. For relative quantification, the expression of all studied miRNAs and endogenous controls is determined simultaneously also in a reference sample, which is used in all performed analyses.

### Example 2

Example 2 describes a general implementation of statistical analysis of data describing the level of selected miRNAs.

Due to the non-normal distribution of data according to the Shapiro-Wilk test, non-parametric tests are used to evaluate the experimental data. Gene expression of miRNAs is compared between individual groups using the Mann-Whitney test and in case of more than 2 compared groups using the Kruskal-Wallis test followed by a post-hoc analysis. The level of statistical significance is determined on the corrected p-value after applying the Benjamini-Hochberg correction. ROC (Receiver Operating Characteristic) curves are also constructed for the respective miRNAs. The area under the curve, the sensitivity and specificity of individual miRNAs, and the optimal cut-off value (the so-called criterion) are evaluated. Furthermore, the optimal cut-off value and sensitivity of a given miRNA biomarker is determined at 90.0% specificity, which corresponds to information about the percentage of women with increased or decreased expression of a specific miRNA at 10.0% false positive rate (FPR). Furthermore, a combined statistical analysis in the form of logistic regression and ROC analysis is performed in order to select the optimal combination of miRNA biomarkers for the given situation. This application provides the following parameters: area under the curve, sensitivity, specificity, optimal cut-off value, and sensitivity of a given combination of miRNA biomarkers at 90.0% specificity.

### Example 3

Example 3 describes a collection and selection of a suitable set of biological samples for the development of a method for predicting pregnancy complications using cardiovascular miRNAs as biomarkers.

200 μL of whole peripheral venous blood is collected from 4,187 women in 10th-13th week of pregnancy. A cell lysate of leukocytes is prepared by removing erythrocytes and then stored deep-frozen at −80 °C. After the patients give birth, samples from women whose complete state of health during the entire pregnancy is known, including all complications and the course of childbirth, are specifically selected. 80 patients with a physiological course of pregnancy and with a negative result of the first-trimester prenatal screening using an established method utilizing the routine predictive algorithm within a computer application for obstetrics and gynecology databases are selected as a control group. For the study of GDM prediction, 121 patients who were diagnosed with GDM during pregnancy are selected. Blood samples of these selected patients are analyzed following the procedure described in Example 1 and levels of selected 11 miRNAs (miR-1-3p, miR-20a-5p, miR-20b-5p, miR-23a-3p, miR-100-5p, miR-125b-5p, miR-126-3p, miR-181 a-5p, miR-195-5p, miR-499a-5p, miR-574-3p) are determined. For the prediction of GDM with sufficient reliability described in Examples 4-12, more than half of the suitable miRNAs shown in the individual examples are used each time. The highest reliability is achieved by using all suitable miRNAs shown in the individual examples.

### Example 4

Example 4 describes a successful prediction of GDM using selected miRNA markers in a selected sample of patients.

121 monitored patients develop GDM during pregnancy. Blood samples of these selected patients are analyzed following the procedure described in Example 1 and levels of selected 11 miRNAs (miR-1-3p, miR-20a-5p, miR-20b-5p, miR-23a-3p, miR-100-5p, miR-125b-5p, miR-126-3p, miR-181 a-5p, miR-195-5p, miR-499a-5p, miR-574-3p) are determined. Up-regulation of these selected miRNA biomarkers, whose levels exceed the minimum values determined by a statistical analysis for a 10% FPR, is observed in 58 of 121 patients, which corresponds to a successful prediction in 47.9% of cases. Statistical analysis of the obtained data provides the following values of specificity, sensitivity, 95% Cl, and criterion:

Combined screening of GDM (11 selected miRNA)

| Area Under the ROC Curve (AUC) | 0.742 | Criterion | >0.585044312 |
|---|---|---|---|
| Standard Error | 0.0345 | Sensitivity | 63.64 |
| 95% Confidence Interval | 0.676-0.801 | 95% CI | 54.4-72.2 |
| Significance Level P (Area = 0.5) | <0.0001 | Specificity | 78.75 |
| Estimated sensitivity at fixed specificity | | 95% CI | 68.2-87.1 |
| Specificity | 90.00 | +LR | 2.99 |
| Sensitivity | 47.93 | 95% CI | 1.9-4.7 |
| 95% Confidence Interval | 35.54-61.98 | −LR | 0.46 |
| Criterion | >0.705293056 | 95% CI | 0.4-0.6 |

CI = Confidence Interval
LR = Likelihood Ratio

### Example 5

Example 5 describes a successful prediction of GDM using selected miRNA markers and basic clinical characteristics of the mother in a selected sample of patients.

121 monitored patients develop GDM during pregnancy. Blood samples of these selected patients are analyzed following the procedure described in Example 1 and levels of selected 11 miRNAs (miR-1-3p, miR-20a-5p, miR-20b-5p, miR-23a-3p, miR-100-5p, miR-125b-5p, miR-126-3p, miR-181 a-5p, miR-195-5p, miR-499a-5p, miR-574-3p) are determined. Up-regulation of these selected miRNA biomarkers and values of three basic clinical characteristics of the mother (age in early pregnancy, BMI in early pregnancy, infertility treatment with assisted reproduction methods), whose levels exceed the minimum values determined by a statistical analysis for a 10% FPR, is observed in 84 of 121 patients, which corresponds to a successful prediction in 69.4% of cases. Statistical analysis of the obtained data provides the following values of specificity, sensitivity, 95% Cl, and criterion:

Combined screening of GDM (11 selected miRNA, 3 clinical characteristics)

| Area Under the ROC Curve (AUC) | 0.835 | Criterion | >0.692915478 |
|---|---|---|---|
| Standard Error | 0.0281 | Sensitivity | 67.50 |
| 95% Confidence Interval | 0.777-0.884 | 95% CI | 58.3-75.8 |
| Significance Level P (Area = 0.5) | <0.0001 | Specificity | 92.50 |
| Estimated sensitivity at fixed specificity | | 95% CI | 84.4-97.2 |
| Specificity | 90.00 | +LR | 9.00 |
| Sensitivity | 69.17 | 95% CI | 4.1-19.6 |
| 95% Confidence Interval | 55.83-78.33 | −LR | 0.35 |
| Criterion | >0.677092674 | 95% CI | 0.3-0.5 |

CI = Confidence Interval
LR = Likelihood Ratio

### Example 6

Example 6 describes a successful prediction of GDM using selected miRNA markers and clinical characteristics of the mother in a selected sample of patients.

121 monitored patients develop GDM during pregnancy. Blood samples of these selected patients are analyzed following the procedure described in Example 1 and levels of selected 11 miRNAs (miR-1-3p, miR-20a-5p, miR-20b-5p, miR-23a-3p, miR-100-5p, miR-125b-5p, miR-126-3p, miR-181 a-5p, miR-195-5p, miR-499a-5p, miR-574-3p) are determined. Up-regulation of these selected miRNA biomarkers and values of seven clinical characteristics of the mother (age in early pregnancy, BMI in early pregnancy, infertility treatment with assisted reproduction methods, maternal history of miscarriage, presence of thrombophilic gene mutations, positive result of first-trimester prenatal screening for early preeclampsia and spontaneous preterm birth before the 34th week of gestation and fetal growth restriction before the 37th week of gestation using an established method of a predictive routine algorithm within a computer application for obstetrics and gynecology databases, the incidence of diabetes mellitus in close first-degree relatives), whose levels exceed the minimum values determined by a statistical analysis for a 10% FPR, is observed in 88 of 121 patients, which corresponds to a successful prediction in 72.7% of cases. Statistical analysis of the obtained data provides the following values of specificity, sensitivity, 95% Cl, and criterion:

Combined screening of GDM (11 selected miRNA, 7 clinical characteristics)

| Area Under the ROC Curve (AUC) | 0.869 | Criterion | >0.65721512 |
|---|---|---|---|
| Standard Error | 0.0246 | Sensitivity | 72.50 |
| 95% Confidence Interval | 0.815-0.913 | 95% CI | 63.6-80.3 |
| Significance Level P (Area = 0.5) | <0.0001 | Specificity | 90.00 |
| Estimated sensitivity at fixed specificity | | 95% CI | 81.2-95.6 |
| Specificity | 90.00 | +LR | 7.25 |
| Sensitivity | 72.50 | 95% CI | 3.7-14.1 |
| 95% Confidence Interval | 60.83-83.33 | −LR | 0.31 |
| Criterion | >0.65721512 | 95% CI | 0.2-0.4 |

CI = Confidence Interval
LR = Likelihood Ratio

### Example 7

Example 7 describes a successful prediction of a milder form of GDM requiring only dietary modifications using selected miRNA markers in a selected sample of patients.

101 monitored patients develop a milder form of GDM requiring only dietary modifications during pregnancy. Blood samples of these selected patients are analyzed following the procedure described in Example 1 and levels of selected 8 miRNAs (miR-1-3p, miR-20a-5p, miR-20b-5p, miR-100-5p, miR-125b-5p, miR-195-5p, miR-499a-5p, miR-574-3p) are determined. Up-regulation of these selected miRNA biomarkers, whose levels exceed the minimum values determined by a statistical analysis for a 10% FPR, is observed in 35 of 101 patients, which corresponds to a successful prediction in 34.7% of cases. Statistical analysis of the obtained data provides the following values of specificity, sensitivity, 95% Cl, and criterion:

Combined screening of a milder form of GDM requiring
only dietary modifications (8 selected miRNA)

| Area Under the ROC Curve (AUC) | 0.691 | Criterion | >0.498016311 |
|---|---|---|---|
| Standard Error | 0.0393 | Sensitivity | 72.28 |
| 95% Confidence Interval | 0.618-0.758 | 95% CI | 62.5-80.7 |
| Significance Level P (Area = 0.5) | <0.0001 | Specificity | 60.00 |
| Estimated sensitivity at fixed specificity | | 95% CI | 48.4-70.8 |
| Specificity | 90.00 | +LR | 1.81 |
| Sensitivity | 34.65 | 95% CI | 1.3-2.4 |
| 95% Confidence Interval | 9.90-48.51 | −LR | 0.46 |
| Criterion | >0.658940555 | 95% CI | 0.3-0.7 |

CI = Confidence Interval
LR = Likelihood Ratio

### Example 8

Example 8 describes a successful prediction of a milder form of GDM requiring only dietary modifications using selected miRNA markers and basic clinical characteristics of the mother in a selected sample of patients.

101 monitored patients develop a milder form of GDM requiring only dietary modifications during pregnancy. Blood samples of these selected patients are analyzed following the procedure described in Example 1 and levels of selected 8 miRNAs (miR-1-3p, miR-20a-5p, miR-20b-5p, miR-100-5p, miR-125b-5p, miR-195-5p, miR-499a-5p, miR-574-3p) are determined. Up-regulation of these selected miRNA biomarkers and values of three basic clinical characteristics of the mother (age in early pregnancy, BMI in early pregnancy, infertility treatment with assisted reproduction methods), whose levels exceed the minimum values determined by a statistical analysis for a 10% FPR, is observed in 51 of 101 patients, which corresponds to a successful prediction in 50.5% of cases. Statistical analysis of the obtained data provides the following values of specificity, sensitivity, 95% Cl, and criterion:

Combined screening of a milder form of GDM requiring only dietary modifications

| Area Under the ROC Curve (AUC) | 0.784 | Criterion | >0.642596346 |
|---|---|---|---|
| Standard Error | 0.0336 | Sensitivity | 61.39 |
| 95% Confidence Interval | 0.717-0.842 | 95% CI | 51.2-70.9 |
| Significance Level P (Are a= 0.5) | <0.0001 | Specificity | 87.50 |
| Estimated sensitivity at fixed specificity | | 95% CI | 78.2-93.8 |
| Specificity | 90.00 | +LR | 4.91 |
| Sensitivity | 50.50 | 95% CI | 2.7-8.9 |
| 95% Confidence Interval | 31.68-69.31 | −LR | 0.44 |
| Criterion | >0.704912418 | 95% CI | 0.3-0.6 |

CI = Confidence Interval
LR = Likelihood Ratio

### Example 9

Example 9 describes a successful prediction of a milder form of GDM requiring only dietary modifications using selected miRNA markers and clinical characteristics of the mother in a selected sample of patients.

101 monitored patients develop a milder form of GDM requiring only dietary modifications during pregnancy. Blood samples of these selected patients are analyzed following the procedure described in Example 1 and levels of selected 8 miRNAs (miR-1-3p, miR-20a-5p, miR-20b-5p, miR-100-5p, miR-125b-5p, miR-195-5p, miR-499a-5p, miR-574-3p) are determined. Up-regulation of these selected miRNA biomarkers and values of seven clinical characteristics of the mother (age in early pregnancy, BMI in early pregnancy, infertility treatment with assisted reproduction methods, maternal history of miscarriage, presence of thrombophilic gene mutations, positive result of first-trimester prenatal screening for early preeclampsia and spontaneous preterm birth before the 34th week of gestation and fetal growth restriction before the 37th week of gestation using an established method of a predictive routine algorithm within a computer application for obstetrics and gynecology databases, the incidence of diabetes mellitus in close first-degree relatives), whose levels exceed the minimum values determined by a statistical analysis for a 10% FPR, is observed in 57 of 101 patients, which corresponds to a successful prediction in 56.4% of cases. Statistical analysis of the obtained data provides the following values of specificity, sensitivity, 95% Cl, and criterion:

Combined screening of a milder form of GDM requiring only dietary modifications
(8 selected miRNA, 7 clinical characteristics)

| Area Under the ROC Curve (AUC) | 0.835 | Criterion | >0.513780486 |
|---|---|---|---|
| Standard Error | 0.0295 | Sensitivity | 77.23 |
| 95% Confidence Interval | 0.773-0.886 | 95% CI | 67.8-85.0 |
| Significance Level P (Area = 0.5) | <0.0001 | Specificity | 78.75 |
| Estimated sensitivity at fixed specificity | | 95% CI | 68.2-87.1 |
| Specificity | 90.00 | +LR | 3.63 |
| Sensitivity | 56.44 | 95% CI | 2.4-5.6 |
| 95% Confidence Interval | 43.56-73.22 | −LR | 0.29 |
| Criterion | >0.679838192 | 95% CI | 0.2-0.4 |

CI = Confidence Interval
LR = Likelihood Ratio

### Example 10

Example 10 describes a successful prediction of a more severe form of GDM requiring therapeutic intervention in addition to dietary modifications using selected miRNA markers in a selected sample of patients.

20 monitored patients develop a more severe form of GDM requiring therapeutic intervention in addition to dietary modifications during pregnancy. Blood samples of these selected patients are analyzed following the procedure described in Example 1 and levels of selected 3 miRNAs (miR-20a-5p, miR-20b-5p, miR-195-5p) are determined. Up-regulation of these selected miRNA biomarkers, whose levels exceed the minimum values determined by a statistical analysis for a 10% FPR, is observed in 6 of 20 patients, which corresponds to a successful prediction in 30.0% of cases. Statistical analysis of the obtained data provides the following values of specificity, sensitivity, 95% Cl, and criterion:

Combined screening of a more severe form of GDM requiring therapeutic
intervention in addition to dietary modifications (3 selected miRNA)

| Area Under the ROC Curve (AUC) | 0.731 | Criterion | >0.198733282 |
|---|---|---|---|
| Standard Error | 0.0578 | Sensitivity | 65.00 |
| 95% Confidence Interval | 0.633-0.815 | 95% CI | 40.8-84.6 |
| Significance Level P (Area = 0.5) | <0.0001 | Specificity | 73.75 |
| Estimated sensitivity at fixed specificity | | 95% CI | 62.7-83.0 |
| Specificity | 90.00 | +LR | 2.48 |
| Sensitivity | 30.00 | 95% CI | 1.5-4.0 |
| 95% Confidence Interval | 10.00-55.00 | −LR | 0.47 |
| Criterion | >0.251230974 | 95% CI | 0.3-0.9 |

CI = Confidence Interval
LR = Likelihood Ratio

### Example 11

Example 11 describes a successful prediction of a more severe form of GDM requiring therapeutic intervention in addition to dietary modifications using selected miRNA markers and basic clinical characteristics of the mother in a selected sample of patients.

20 monitored patients develop a more severe form of GDM requiring therapeutic intervention in addition to dietary modifications during pregnancy. Blood samples of these selected patients are analyzed following the procedure described in Example 1 and levels of selected 3 miRNAs (miR-20a-5p, miR-20b-5p, miR-195-5p) are determined. Up-regulation of these selected miRNA biomarkers and values of three basic clinical characteristics of the mother (age in early pregnancy, BMI in early pregnancy, infertility treatment with assisted reproduction methods), whose levels exceed the minimum values determined by a statistical analysis for a 10% FPR, is observed in 16 of 20 patients, which corresponds to a successful prediction in 80.0% of cases. Statistical analysis of the obtained data provides the following values of specificity, sensitivity, 95% Cl, and criterion:

Combined screening of a more severe form of GDM requiring therapeutic
intervention in addition to dietary modifications
(3 selected miRNA, 3 clinical characteristics)

| Area Under the ROC Curve (AUC) | 0.949 | Criterion | >0.191259321 |
|---|---|---|---|
| Standard Error | 0.0229 | Sensitivity | 89.47 |
| 95% Confidence Interval | 0.886-0.983 | 95% CI | 66.9-98.7 |
| Significance Level P (Area = 0.5) | <0.0001 | Specificity | 86.25 |
| Estimated sensitivity at fixed specificity | | 95% CI | 76.7-92.9 |
| Specificity | 90.00 | +LR | 6.51 |
| Sensitivity | 78.95 | 95% CI | 3.7-11.5 |
| 95% Confidence Interval | 50.78-94.74 | −LR | 0.12 |
| Criterion | >0.243735973 | 95% CI | 0.03-0.5 |

CI = Confidence Interval
LR = Likelihood Ratio

### Example 12

Example 12 describes a successful prediction of a more severe form of GDM requiring therapeutic intervention in addition to dietary modifications using selected miRNA markers and clinical characteristics of the mother in a selected sample of patients.

20 monitored patients develop a more severe form of GDM requiring therapeutic intervention in addition to dietary modifications during pregnancy. Blood samples of these selected patients are analyzed following the procedure described in Example 1 and levels of selected 3 miRNAs (miR-20a-5p, miR-20b-5p, miR-195-5p) are determined. Up-regulation of these selected miRNA biomarkers and values of seven clinical characteristics of the mother (age in early pregnancy, BMI in early pregnancy, infertility treatment with assisted reproduction methods, maternal history of miscarriage, presence of thrombophilic gene mutations, positive result of first-trimester prenatal screening for early preeclampsia and spontaneous preterm birth before the 34th week of gestation and fetal growth restriction before the 37th week of gestation using an established method of a predictive routine algorithm within a computer application for obstetrics and gynecology databases, the incidence of diabetes mellitus in close first-degree relatives), whose levels exceed the minimum values determined by a statistical analysis for a 10% FPR, is observed in 18 of 20 patients, which corresponds to a successful prediction in 90.0% of cases. Statistical analysis of the obtained data provides the following values of specificity, sensitivity, 95% Cl, and criterion:

Combined screening of a more severe form of GDM requiring
therapeutic intervention in addition to dietary modifications
(3 selected miRNA, 7 clinical characteristics)

| Area Under the ROC Curve (AUC) | 0.957 | Criterion | >0.211657025 |
|---|---|---|---|
| Standard Error | 0.0197 | Sensitivity | 89.47 |
| 95% Confidence Interval | 0.897-0.988 | 95% CI | 66.9-98.7 |
| Significance Level P (Area = 0.5) | <0.0001 | Specificity | 90.00 |
| Estimated sensitivity at fixed specificity | | 95% CI | 81.2-95.6 |
| Specificity | 90.00 | +LR | 8.95 |
| Sensitivity | 89.47 | 95% CI | 4.6-17.6 |
| 95% Confidence Interval | 68.42-100.00 | −LR | 0.12 |
| Criterion | >0.211657025 | 95% CI | 0.03-0.4 |

CI = Confidence Interval
LR = Likelihood Ratio

### INDUSTRIAL APPLICABILITY

Method of prediction of gestational diabetes mellitus based on the expression profile of cardiovascular miRNAs is industrially applicable in the clinical practice of gynecology and obstetrics in the laboratory analysis of collected samples of biological material.

1. Method of prediction of gestational diabetes mellitus, characterized in that pregnant woman is screened to determine the expression profile of two or more miRNAs in whole peripheral venous blood collected between the 10th and the 13th gestational week to predict gestational diabetes mellitus by their up-regulation, whereas said two or more miRNAs are selected from the group miR-1-3p, miR-20a-5p, miR-20b-5p, miR-23a-3p, miR-100-5p, miR-125b-5p, miR-126-3p, miR-181 a-5p, miR-195-5p, miR-499a-5p, miR-574-3p.

2. Method according to claim 1, characterized in that the screened miRNAs for prediction of a milder form of gestational diabetes mellitus are miR-1-3p, miR-20a-5p, miR-20b-5p, miR-100-5p, miR-125b-5p, miR-195-5p, miR-499a-5p, and miR-574-3p.

3. Method according to claim 1, characterized in that the screened miRNAs for prediction of a more severe form of gestational diabetes mellitus are miR-20a-5p, miR-20b-5p, and miR-195-5p.
